(19) United States
(12) Patent Application Publication
(10) Pub. No.: US 2025/0114553 A1
(43) Pub. Date: Apr. 10, 2025

(54) APPARATUS TO MODERATE BEHAVIOR WITH VISUAL, AUDITORY, AND BREATHING REGULATION

(71) Applicant: Webe kälm LLC, Reno, NV (US)

(72) Inventors: Ryan Richard Dubach, Reno, NV (US); Matthew Aaron Lederman, Los Angeles, CA (US); Alona Pulde Lederman, Los Angeles, CA (US)

(21) Appl. No.: 18/907,972

(22) Filed: Oct. 7, 2024

Related U.S. Application Data
(60) Provisional application No. 63/542,635, filed on Oct. 5, 2023.

(51) Int. Cl. A61M 16/08; A61M 21/00; A61M 21/02

(52) U.S. Cl. CPC A61M 16/08; A61M 21/02; A61M 2021/0027; A61M 2205/583

(57) ABSTRACT

A living subject or patient, may blow into a primary air passageway within a breath conduit positioned to receive the patient's breath and/or air into the primary air passageway. The air pressure from the breath conduit sends an indicator device, such as a lightweight ball, aloft within the secondary air passageway. The air pressure further sends air through a plurality of air openings that generate white noise during use. The air pressure, the visual stimulation of watching the ball float, and the white noise are used to provide a therapeutic effect to a person having anxiety and avoid hyperventilation.

### CROSS REFERENCE TO RELATED APPLICATIONS

This application claims the benefit of priority to U.S. Provisional Application No. 63/542,635, filed Oct. 5, 2023, which is incorporated by reference herein in its entirety.

### BACKGROUND

Breathing regulation is a known technique for relief from anxiety, temper tantrums, panic attacks, and other symptoms of mental and/or emotional distress in a living subject. Breathing regulation allows for a patient to slow the breathing and respiration processes to bring a sense of calmness to the patient's mind. Breathing regulation also regulates blood chemistry changes that can be caused by hyperventilation, carbon dioxide levels in the blood, oxygen levels in the blood, hormone levels including cortisol and adrenaline, and overall neurotransmitter functions in the brain.

Even though breathing regulation has been known to ease the symptoms of distress for greater mindfulness in stressful situations, very few devices are available to assist patients with an efficient way achieve the full health benefits that are available with respiration control. This is due in part to the fact that patients are expected to control their breathing on their own while their brains are under the strong influences of stress, blood chemistry irregularities, and hormonal effects of anxiety. Whether the stress and anxiety is caused by anger, lack of emotional control, outside stressors, or even chronic negative thought processes, a need exists in the fields of psychology, wellness, and mindfulness for a new approach to bringing calmness to a person for a sufficient amount of time to re-assess behaviors. There is also a need for new approaches to implement more sensory adaptations, such as visible stimuli and audible stimuli, simultaneously with breathing regulation for maximum benefit.

### BRIEF SUMMARY OF THE DISCLOSURE

A method and apparatus according to this disclosure incorporates three sensory inputs to help a patient experience more emotional control during times of stress. The apparatus and method of this disclosure incorporate visual inputs, audible inputs, and breathing regulation processes to move a patient from a highly anxious state to a calmer state to allow the patient to implement additional calming techniques to their current experience.

In one embodiment, an apparatus to regulate breathing includes a breath conduit 136 defining a primary air passageway 130 there through. The apparatus further includes an indicator adaptor having a flange portion and a receiving slot portion, wherein the receiving slot portion is configured to connect to the breath conduit. The indicator adaptor defines an air chamber in fluid communication with the primary air passageway of the breath conduit. The indicator adaptor further defines a plurality of air outlets 180 in fluid communication with the air chamber; wherein the flange portion of the indicator adaptor defines an adaptor air outlet 165 in fluid communication with the air chamber. An indicator housing 160 defines a secondary air passageway 155 there through, wherein the indicator housing fits within the flange portion of the indicator adaptor, and wherein the indicator housing defines an air inlet 140 in fluid communication with the adaptor air outlet 165 and the air chamber 170 of the indicator adaptor. An indicator device 145 is positioned within the indicator housing 160, and the indicator device is configured to traverse the secondary air passageway 155 with air pressure from the air inlet 140.

In another embodiment, a method of regulating breathing by living subject includes blowing air from the living subject through a primary air passageway within a breath conduit; constricting the air flow through the primary air passageway to increase an air pressure force therein; collecting the air flow within a partially enclosed air chamber in fluid communication with the primary air passageway of the breath conduit; pushing a portion of the air flow through a plurality of air outlets in fluid communication with the air chamber to change air pressure forces within the air chamber; pushing second portion of the air flow from the air chamber through an adaptor air outlet; pushing the second portion of the air flow through a secondary air passageway of an indicator housing; and moving an indicator device positioned within the secondary air passageway of the indicator housing.

### BRIEF DESCRIPTION OF THE DRAWINGS

These and other features, aspects, and advantages of the present invention will become apparent from the following description and the accompanying exemplary embodiments shown in the drawings, which are briefly described below.

FIG. 1 is a side plan view of a cross section of a breathing regulation apparatus according to this disclosure.

FIG. 2 is a side perspective view of a breathing regulation apparatus according to this disclosure.

FIG. 3 is a side plan view of a breathing regulation apparatus in a vertical position according to this disclosure.

FIG. 4 is a rear perspective side view of a breathing regulation apparatus in a vertical position according to this disclosure.

FIG. 5 is a rear view of a breathing regulation apparatus in a vertical position according to this disclosure.

FIG. 6 is a top perspective view of a breathing regulation apparatus according to this disclosure.

FIG. 7 is a side perspective view of a breathing regulation apparatus according to this disclosure.

FIG. 8 is a top perspective view of a breathing regulation apparatus according to this disclosure.

FIG. 9 is a front plan view of a breathing regulation apparatus according to this disclosure with the air outlet openings of an indicator adaptor facing forward and the indicator housing extending downward along an axis perpendicular to the indicator adaptor.

### DETAILED DESCRIPTION

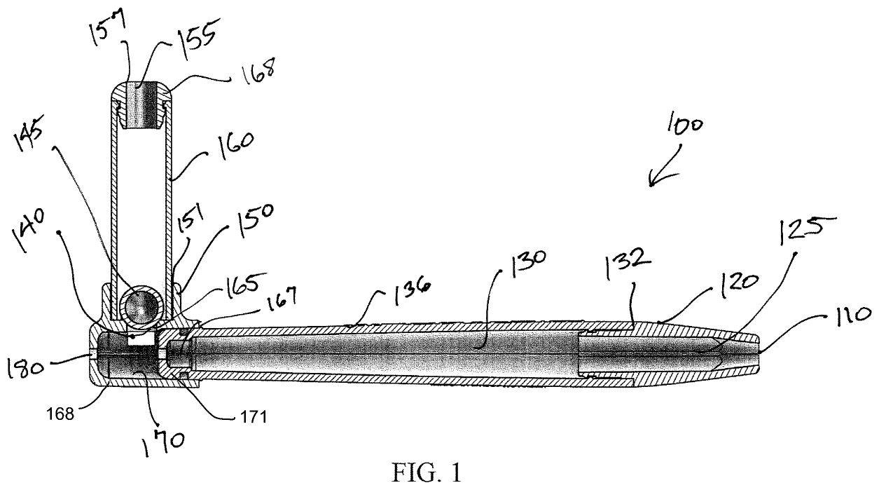

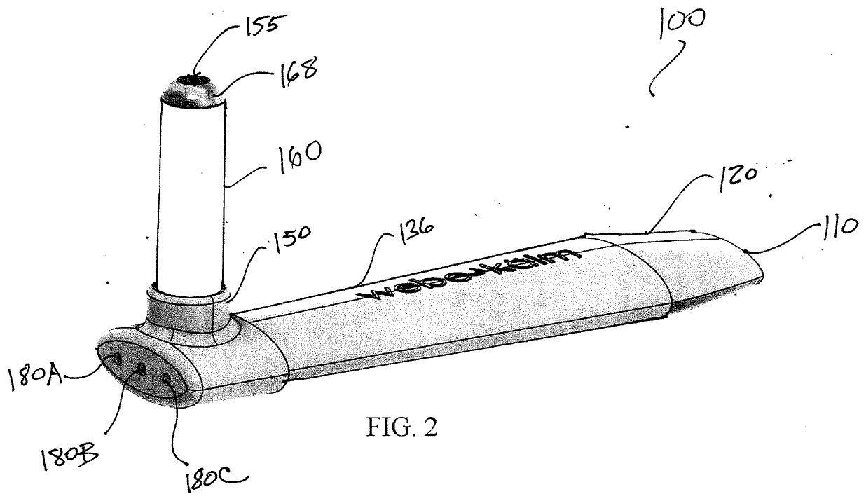

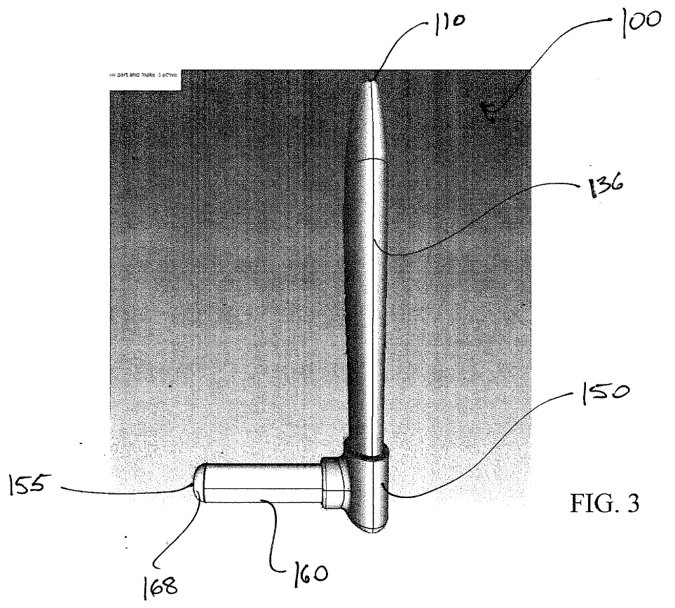

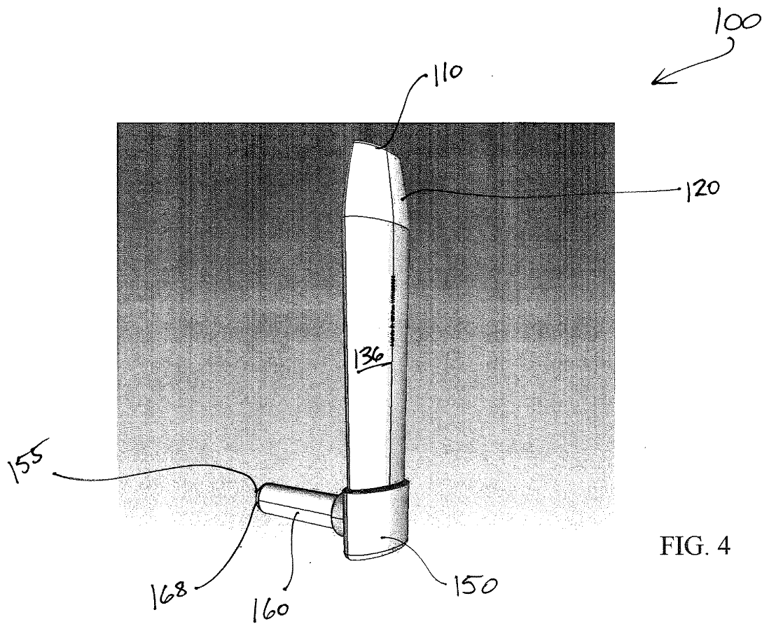

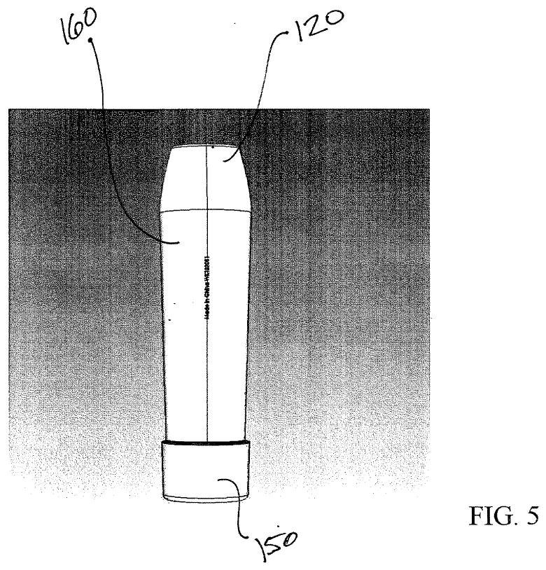

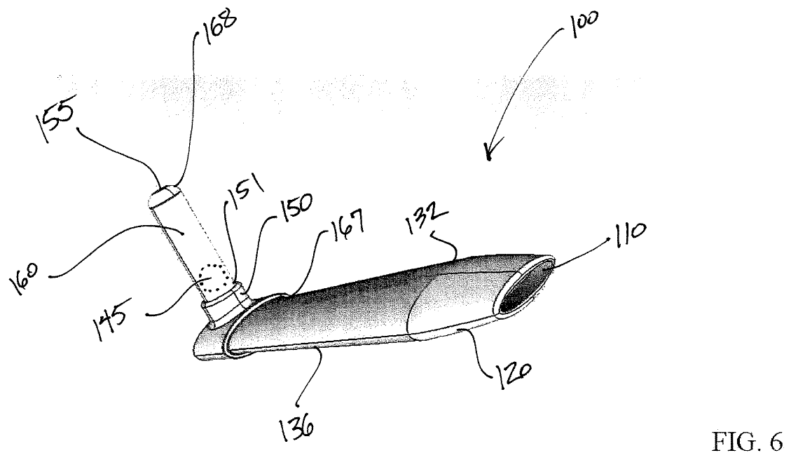

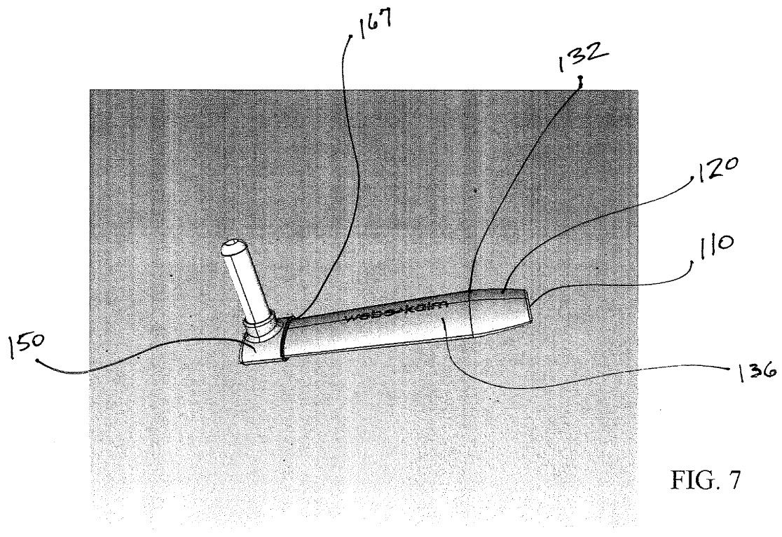

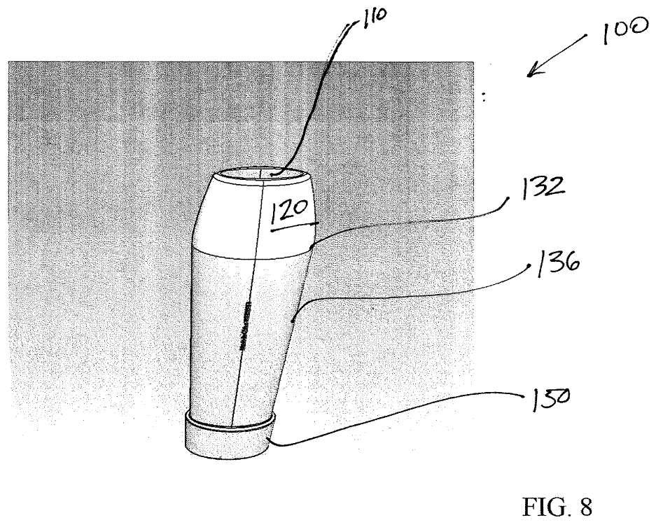

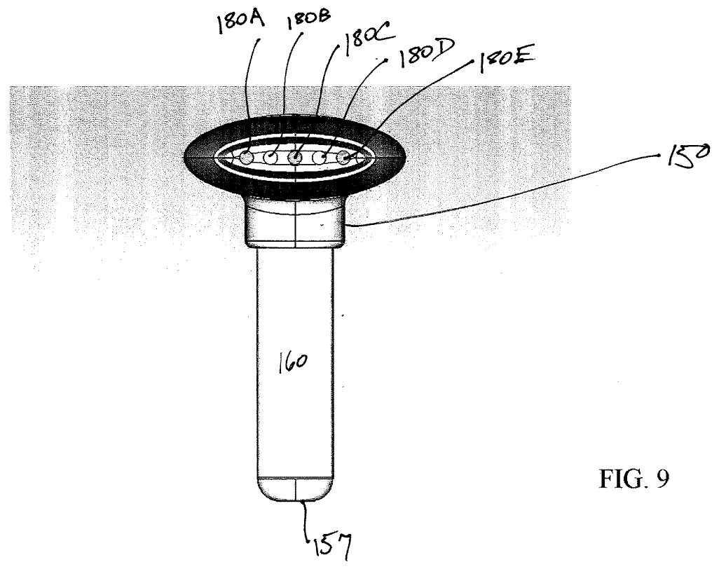

In one embodiment illustrated in FIGS. 1-9 attached here, an apparatus 100 to regulate breathing includes a breath conduit 136 defining a primary air passageway 130 there through. An indicator adaptor 150 that includes a flange portion 151 and a receiving slot portion 168 is adapted or configured to attach to the breath conduit. The receiving slot portion 168 of the indicator adaptor 150 is configured to connect to the breath conduit, wherein the indicator adaptor defines an air chamber 170 in fluid communication with the primary air passageway of the breath conduit. The indicator adaptor further defines a plurality of air outlets 180 in fluid communication with the air chamber 170. The flange portion 151 of the indicator adaptor defines an adaptor air outlet 165 in fluid communication with the air chamber. An indicator housing 160 defines a secondary air passageway 155 there through, wherein the indicator housing fits within the flange portion of the indicator adaptor, and wherein the indicator housing defines an air inlet 140 in fluid communication with the adaptor air outlet 165 and the air chamber 170 of the indicator adaptor. An indicator device 145 is positioned within the indicator housing 160, and the indicator device is configured to traverse the secondary air passageway 155 with air pressure from the air inlet 140. The breath conduit 136 may optionally include a mouthpiece 120 defining a breath inlet 110 in fluid communication with the primary air passageway 130 of the breath conduit 136. In some embodiments, the breath conduit defines a breath outlet 167 for air to exit the primary air passageway 130 and enter the air chamber 170. In non-limiting embodiments, the primary air passageway is tapered to increase air pressure within the breath conduit 136 when air is pushed through the primary air passageway 130.

The air chamber 170 of the indicator adaptor 150 is defined by the slot portion 168 of the indicator adaptor and one end 171 of the breath conduit 136 that fits within the slot portion 168. Upon air blowing through the primary air passageway 130, air pressure increases above atmospheric pressure within the air chamber 170 and the secondary air passageway 155 of the indicator housing 160, and wherein increased air pressure moves the indicator device 145 within the secondary air passageway 155. In non-limiting embodiments, the indicator housing has a clear portion making the indicator device 145 visible within the secondary air passageway 155. The indicator device 145 is a ball or any other item that is so dimensioned to be seated on the adaptor air outlet 165 and within the secondary air passageway 155, the indicator device 145 receiving increased air pressure from the air chamber 170 thereon. In some non-limiting embodiments, constrictions at the breath outlet of the primary air passageway 130, additional constrictions at the plurality of air outlets 180, and the indicator device 145 proximate the adaptor air outlet 165 partially control air pressure within the apparatus 100.

The apparatus 100 further includes a mouth piece, whether detachable or integral to the apparatus 100, defining a breath inlet 110 in fluid communication with the primary air passageway 155 of the breath conduit, wherein the primary air passageway 130 comprises a back pressure force in the direction of the mouth piece, helping a user to control the respiration rate during use. In some embodiments, the mouth piece 120, the breath conduit 130, the indicator adaptor 150, and the indicator housing 160 are modular components that connect and disconnect from each other. In other embodiments, the components of the apparatus 100 are integrally manufactured in one piece.

In one non-limiting embodiment, the plurality of air outlets 180 are in positions to allow for vibrating air, subject to pressure differentials in the air chamber 170, to exit the apparatus 100 and make audible white noise at air pressure above atmospheric air pressure. The audible white noise may be part of a therapy that allows a user to regulate breathing and focus on the sound of the white noise to help with mindfulness and calming experiences (i.e., to help get the patient's mind off of the stressors at hand). In the embodiments, the primary air passageway 130, the air chamber 170, the plurality of air outlets 180, and the secondary air passageway 160 are so dimensioned to provide an air pressure that is sufficient to move the indicator device 145 within the secondary air passageway 155.

In use of the apparatus 130 described above, a living subject or patient, may blow into the primary air passageway 130 with the breath conduit 136 positioned to receive the patient's breath and/or air into the primary air passageway 130. Ultimately, this breath will send the indicator device 145 aloft within the secondary air passageway 155. The indicator device 145 is shown in the figures as being a lightweight or hollow ball, but this shape is not limiting of the embodiments disclosed herein. The indicator housing 160 can be configured with numerous kinds of indicator devices that show the user and other viewers that the user is pushing a single breath into the air chamber 170 and holding the air pressure within the apparatus 100 sufficiently to keep the indicator device 145, such as a ball, aloft within the indicator housing 160. Holding the air pressure through the apparatus with a single breath from the user's mouth is one way to slow and regulate breathing rates. Holding the ball aloft within the secondary air passageway is visible, and the user can use the indicator device as a visible sign that the air pressure is at a therapeutic level because the ball is staying aloft. The use of a ball is just an example, and other indicator devices, including lights controlled by air pressure or other shaped items within the housing can be used to show that the air pressure pushed through the apparatus is sufficient. Therapeutic use of the apparatus may include timing the state of the ball/indicator device and encouraging the user to hold the ball aloft for a certain period of time. A timer can be included in the apparatus, or a separate clock may be used to time a user's breath of air holding the indicator device aloft. The visual aspect of watching the ball float within the indicator housing 160 also provides a therapeutic effect because it gives the user something to concentrate on besides the stressors at hand. Holding the ball aloft with air pressure originating from the mouth piece may be simultaneously occurring while the air outlets 180 are providing white noise that is audible. In this way, the air pressure and back pressure within the apparatus 100 regulate the pace of respiration, the visual stimulation of watching where the ball/indicator device floating, and the audio stimulation of the white noise can provide sensory inputs that help a patient feel a sense of wellness and calm down.

Accordingly, a method of regulating breathing by living subject includes steps to achieve the desired calming effect with the apparatus 100. The method includes blowing air from the living subject through a primary air passageway 130 within a breath conduit 136; constricting the air flow through the primary air passageway to increase an air pressure force therein; collecting the air flow within a partially enclosed air chamber 170 in fluid communication with the primary air passageway of the breath conduit; pushing a portion of the air flow through a plurality of air outlets 180 in fluid communication with the air chamber 170 to increase air pressure forces within the air chamber; pushing second portion of the air flow from the air chamber through an adaptor air outlet 165; pushing the second portion of the air flow through a secondary air passageway 155 of an indicator housing 160; and moving an indicator device positioned within the secondary air passageway of the indicator housing. The method may further include viewing the indicator devices, such as the ball in the figures, moving through the secondary air passageway and being held in certain positions by air pressure from a user's breath. The method further includes generating white noise sounds with a plurality of air outlets.

For purposes of this disclosure, the term “coupled” or “connected” means the joining of two components (electrical, mechanical, or magnetic) directly or indirectly to one another. Such joining may be stationary in nature or movable in nature. Such joining may be achieved with the two components (electrical or mechanical) and any additional intermediate members being integrally defined as a single unitary body with one another or with the two components or the two components and any additional member being attached to one another. Such joining may be permanent in nature or temporary in nature. References 132 and 167 show such couplings.

The present disclosure has been described with reference to example embodiments, however persons skilled in the art will recognize that changes may be made in form and detail without departing from the spirit and scope of the claimed subject matter. For example, although different example embodiments may have been described as including one or more features providing one or more benefits, it is contemplated that the described features may be interchanged with one another or alternatively be combined with one another in the described example embodiments or in other alternative embodiments. Because the technology of the present disclosure is relatively complex, not all changes in the technology are foreseeable. The present disclosure described with reference to the example embodiments and set forth in the following claims is manifestly intended to be as broad as possible. For example, unless specifically otherwise noted, the claims reciting a single particular element also encompass a plurality of such particular elements.

It is also important to note that the construction and arrangement of the elements of the system as shown in the preferred and other exemplary embodiments is illustrative only. Although only a certain number of embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements shown as multiple parts may be integrally formed, the operation of the assemblies may be reversed or otherwise varied, the length or width of the structures and/or members or connectors or other elements of the system may be varied, the nature or number of adjustment or attachment positions provided between the elements may be varied. It should be noted that the elements and/or assemblies of the system may be constructed from any of a wide variety of materials that provide sufficient strength or durability.

Accordingly, all such modifications are intended to be included within the scope of the present disclosure. Other substitutions, modifications, changes and omissions may be made in the design, operating conditions and arrangement of the preferred and other exemplary embodiments without departing from the spirit of the present subject matter.

Computer systems may be used in manufacturing the apparatus of this disclosure. Computer systems may be utilized to diagnose a patient's condition and set the dimensions of an apparatus that is prescribed for a patient's condition (i.e., a computer may be used to evaluate the most therapeutic air pressure levels within each section of the apparatus and optimize breathing rates for a given patient's physiology). In other words, the apparatuses and methods of this disclosure may incorporate the use of computers to diagnose a patient and prescribe a custom apparatus claimed below with features, operating conditions and dimensions that suit an individual patient's needs. Computer systems (and/or their associated structures) may also include suitable interfaces for receiving. transmitting, and/or otherwise communicating data from the apparatus 100 in a network environment.

1. An apparatus to regulate breathing, comprising:
a breath conduit defining a primary air passageway there through;
an indicator adaptor comprising a flange portion and a receiving slot portion, wherein the receiving slot portion is configured to connect to the breath conduit,
wherein the indicator adaptor defines an air chamber in fluid communication with the primary air passageway of the breath conduit;
wherein the indicator adaptor further defines a plurality of air outlets in fluid communication with the air chamber;
wherein the flange portion of the indicator adaptor defines an adaptor air outlet in fluid communication with the air chamber;
an indicator housing defining a secondary air passageway there through, wherein the indicator housing fits within the flange portion of the indicator adaptor, and wherein the indicator housing defines an air inlet in fluid communication with the adaptor air outlet and the air chamber of the indicator adaptor; and
an indicator device positioned within the indicator housing, the indicator device configured to traverse the secondary air passageway with air pressure from the air inlet.

2. The apparatus of claim 1, wherein the breath conduit comprises a mouthpiece defining a breath inlet in fluid communication with the primary air passageway of the breath conduit.

3. The apparatus of claim 1, wherein the breath conduit defines a breath outlet for air to exit the primary air passageway and enter the air chamber.

4. The apparatus of claim 1, wherein the primary air passageway is tapered to increase air pressure within the breath conduit when air is pushed through the primary air passageway.

5. The apparatus of claim 1, wherein the air chamber of the indicator adaptor is defined by the slot portion of the indicator adaptor and one end of the breath conduit that fits within the slot portion.

6. The apparatus of claim 5, wherein upon air blowing through the primary air passageway, air pressure increases above atmospheric pressure within the air chamber and the secondary air passageway of the indicator housing, and wherein increased air pressure moves the indicator device within the secondary air passageway.

7. The apparatus of claim 1, wherein the indicator housing has a clear portion making the indicator device visible within the secondary air passageway.

8. The apparatus of claim 1, wherein the indicator device is a ball that is so dimensioned to be seated on the adaptor air outlet and within the secondary air passageway, the indicator device receiving increased air pressure from the air chamber thereon.

9. The apparatus of claim 1, wherein constrictions at the breath outlet of the primary air passageway, additional constrictions at the plurality of air outlets, and the indicator device proximate the adaptor air outlet partially control air pressure within the apparatus.

10. The apparatus of claim 1, further comprising a mouth piece defining a breath inlet in fluid communication with the primary air passageway of the breath conduit, wherein the primary air passageway comprises a back pressure force in the direction of the mouth piece.

11. The apparatus of claim 1, wherein the plurality of air outlets comprise positions of vibrating air exiting the apparatus and making audible white noise at air pressure above atmospheric air pressure.

12. The apparatus of claim 1, wherein the breath conduit, the indicator adaptor, and the indicator adaptor are modular components that connect and disconnect from each other.

13. The apparatus of claim 1, wherein the breath conduit, the indicator adaptor, and the indicator adaptor are components of a unitary one piece device.

14. The apparatus of claim 1, further comprising a cap at a distal end of the indicator housing opposite the air inlet of the indicator housing.

15. The apparatus of claim 1, wherein the primary air passageway, the air chamber, the plurality of air outlets, and the secondary air passageway are so dimensioned to provide an air pressure that is sufficient to move the indicator device within the secondary air passageway.

16. A method of regulating breathing by living subject, the method comprising:
blowing air from the living subject through a primary air passageway within a breath conduit;
constricting the air flow through the primary air passageway to increase an air pressure force therein;
collecting the air flow within a partially enclosed air chamber in fluid communication with the primary air passageway of the breath conduit;
pushing a portion of the air flow through a plurality of air outlets in fluid communication with the air chamber to increase air pressure forces within the air chamber;
pushing second portion of the air flow from the air chamber through an adaptor air outlet;
pushing the second portion of the air flow through a secondary air passageway of an indicator housing; and
moving an indicator device positioned within the secondary air passageway of the indicator housing.

17. The method of claim 16, further comprising viewing the indicator devices as the indicator device moves.

18. The method of claim 17, further comprising generating white noise sounds through the plurality of air outlets.

19. The method of claim 18, wherein the moving the indicator device, viewing the indicator device, and generating white noise happen substantially simultaneously.

20. The method of claim 19, wherein blowing air from the subject comprises a single exhale that is held for a predetermined time period.
